(19) United States
(12) Patent Application Publication
(10) Pub. No.: US 2003/0118538 A1
(43) Pub. Date: Jun. 26, 2003

(54) Sprayable hair gels

(76) Inventors: Thomas Krause, Darmstadt (DE); Anja Duchscherer, Hadamar (DE); Manuela Hannich, Egelsbach (DE); Hildegard Henze, Darmstadt (DE)

Correspondence Address: STRIKER, STRIKER & STENBY, 103 East Neck Road, Huntington, NY, 11743, US

(21) Appl. No.: 10/247,180

(22) Filed: Sep. 19, 2002

(30) Foreign Application Priority Data
Dec. 21, 2001 (DE) 101 63 500.1

(51) Int. Cl. A61K 7/06; A61K 7/11

(52) U.S. Cl. 424/70.16

(57) ABSTRACT

The hair gel composition contains a combination of at least one hair-fixing polymer and at least one polymeric gel former. The polymeric gel formers are homopolymers or copolymers built up from at least one type of monomer, which is an acrylamidoalkylsulfonic acid, a methacrylicamidoalkylsulfonic acid or a salt thereof, and from acrylate polymers with thickening action, which are built up from acrylic acid or methacrylic acid and at least one acrylic or methacrylic acid ester. The hair gel composition is suitable for a non-aerosol spray product for increasing hair luster and/or hold or fixing of the hair.

### BACKGROUND OF THE INVENTION

The subject matter of the present invention includes compositions for hair treatment in gel form, which contain a combination of (A) at least one polymeric gel former, which is selected from the group consisting of homopolymers and copolymers, which are built up from at least one monomer consisting of an acrylamidoalkylsulfonic acid, a methacrylamidoalkylsulfonic acid and/or a salt thereof, and from acrylate polymers with a thickening action, which are built up from acrylic acid or methacrylic acid and at least one acrylic acid ester or methacrylic acid ester, and (B) at least one hair fixing polymer; as well as hair spray products containing these compositions.

Hair gels are used, in order to give human hair hold and to fix it. Usually hair gels contain a combination of gel formers and hair fixing polymers. The hair fixing polymers used for these purposes in cosmetics are usually characterized by good fixing properties in aqueous, alcoholic or aqueous-alcoholic compositions, which fix and hold the hair more or less satisfactorily after application. Frequently these effects are associated with an insufficient or unsatisfactory luster. However a series of additives are known, which are in a position to improve the hair luster, e.g. hydrophobic materials, such as liquid paraffins, isoparaffins, silicone oils or hydrophilic materials, such as multivalent alcohol, especially glycerol or propylene glycol. The known luster-providing agents however frequently have disadvantages in addition to their other desirable properties. They can act as a softener for the fixing polymers that are used and thus decrease the fixing efficiencies. They can be incompatible with the aqueous gel base. They can impair the clarity and transparency of the gel, the rheological properties and thus the application properties. Alternatively then are still insufficient in improving the hair luster.

Gel formulations usually dispensed from tubes disadvantageously cannot be dispensed in measured amounts and cannot be distributed uniformly on the hair. They produce a non-uniform and large load on the hair. Spray products can be distributed in measured amounts more uniformly on the hair. However there are disadvantages to sprayable gels. They can disadvantageously produce a spray pattern, which is too large with a proportion of large spray droplets. Alternatively a spray pump with a very high operating force must be used in order to obtain a satisfactory spray pattern. The spray pattern can also be too fine with undesirably high properties of very small inhalable or lung-entering spray droplets.

### SUMMARY OF THE INVENTION

It is an object of the present invention to avoid the above-described disadvantages of commercially obtainable hair gels, especially to provide a gel-form product, which, on the one hand, produces improved hair fixing with optimum hair luster, while, on the other, has good sprayability and an optimum spray pattern with optimized droplet size distribution during spraying.

These objects and others, which will be made more apparent hereinafter, are attained in a hair gel composition containing a combination of

(A) at least one gel former selected from the group consisting of homopolymers, copolymers and thickening acrylate polymers; wherein the homopolymers and copolymers are built up from at least one monomer, which is an acrylamidoealkylsulfonic acid, methacrylamidoalkylsulfonic acid or a salt thereof, and the thickening acrylate polymers are made from acrylic or methacrylic acid and at least one acrylic or methacrylic acid ester, wherein the acrylate polymers are selected from those acrylate polymers, which provide a viscosity, in 1% aqueous solution, after adjusting pH to 7.5 with sodium hydroxide, in a range of 2000 to 8000 mPa·s (20° C., Brookfield RVT, 20 U/min); and

(B) at least one hair-fixing polymer.

The viscosity of the gel amounts, preferably, to from 100 to 5000 mPa·s, especially preferably from 200 to 1000 mPa·s, and most preferably from 250 to 800 mPa·s, measured as a shear viscosity with Bohlin Rheometer CS, measuring body C25, at a temperature of 25° C. and a shear rate of 50 s⁻¹.

The gel former (A) preferably is used in an amount of from 0.1 to 10, especially preferably from 0.2 to 8, percent by weight, and the hair-fixing polymer (B) is used in an amount of preferably from 0.1 to 15, especially preferably from 0.5 to 10, percent by weight. The hair gel according to the invention provides good hair fixing and an improved hair luster at the same time as providing improve spray properties, when it is sprayed by means of a spraying apparatus.

### Gel Former (A)

The gel former according to the invention is characterized by imparting more luster and more fixing to human hair than the corresponding gel formers of the prior art. A gel former suitable for the invention is a homopolymer or copolymer built up from at least one monomer type selected from acrylamidoalkylsulfonic acid or methacrylamidoalkylsulfonic acid or their salts. Suitable salts are those with ammonium, alkali metal or alkaline earth metal cations. The polymer is preferably built up from monomers of the general formula: H₂C=CH—C(=O)—NH-A-SO₃H or their salts, wherein A represents a divalent C₂- to C₆-hydrocarbon group, preferably C₃- to C₄-hydrocarbon group, especially preferably the —C(CH₃)₂—CH₂— group. This monomer is preferably copolymerized with at least one nonionic, radically copolymerizable monomer, especially a vinyl lactam, especially preferably vinyl pyrrolidone. A gel former with the INCI name ammonium acryloyldimethyltaurate/VP copolymer is especially preferred as the gel former used in the invention. This latter gel former may be obtained commercially as a commercial product under the tradename Aristoflex® AVC.

Additional gel formers, which are suitable, include acrylate polymers with thickening action. These are copolymers made from at least one first monomer, which is acrylic acid and/or methacrylic acid, and at least one second monomer, which is an acrylic acid ester and/or a methacrylic acid ester, especially the simple esters, e.g. the C₁- to C₄-alkyl esters. These polymers have the INCI name, acrylates copolymer. The “thickening acrylates polymers” are those polymers, which provide a viscosity, in 1% aqueous solution after adjusting pH to 7.5, in a range of 2000 to 8000, preferably 3000 to 6000, mPa·s (20° C., Brookfield RVT, 20 U/min). Carbopol®) Aqua SF-1 is a suitable commercially available product.

The acid groups in the gel former that is used are preferably neutralized 50 to 100% by organic or inorganic bases. Suitable neutralization agents are primary or secondary amines, especially aminoalkanols with preferably from 1 to 10 carbon atoms and 1 to 3 hydroxy groups, such as aminomethylpropanol (AMP), triethanolamine, tetrahydroxypropylethylenediamine or monoethanolamine. However ammonia, NaOH, KOH, among others, can be used as the neutralization agent.

### Hair-fixing Polymer (B)

The hair-fixing polymer (B) can be non-ionic, anionic, zwitterionic or amphoteric. It can be a synthetic or a natural polymer. “Natural polymers” are understood to include chemically modified polymers of natural origin. Those polymers are particularly preferred, which have sufficient solubility or dispersibility in water, alcohol or alcohol/water mixtures, in order to be present in completely dissolved form in compositions according to the invention. “Hair-fixing polymers” are understood to be those polymers, which, when used in an amount of from 0.01 to 5% in an aqueous, alcoholic or aqueous-alcoholic solution, are in a position to deposit a polymer film on the hair and to fix the hair.

Suitable synthetic non-ionic polymers include homopolymers or copolymers, which are built up from at least one of the following monomers: vinyl pyrrolidone, vinyl caprolactam, vinyl esters such as vinyl acetate, vinyl alcohol, acrylamide, methacrylamide, alkyl- and dialkylacrylamide, alkyl- and dialkyl-methacrylamide, ialkylaminoalkylmethacrylamide, dialkylaminoalkylacrylamide, alkylacrylate, alkylmethacrylate, propylene glycol or ethylene glycol, wherein the alkyl groups in these monomers preferably have from one to seven carbon atoms, especially preferably from one to three carbon atoms. For example, homopolymers of vinyl caprolactam, of vinyl pyrrolidone or of N-vinylformamide, are especially suitable. Additional suitable synthetic hair-fixing polymers are, e.g., copolymerizates of vinyl pyrrolidone and vinyl acetate; terpolymers of vinyl pyrrolidone, vinyl acetate and vinyl propionate; terpolymers of vinyl pyrrolidone, vinyl caprolactam and dialkylaminoalkyl(meth)acrylate; terpolymers of vinyl pyrrolidone, vinyl caprolactam and dialkylaminoalkyl(meth)acrylamide; polyacrylamide, polyvinyl alcohols, as well as polyethylene glycol/polypropylene glycol copolymers. Nonionic vinyl lactam homopolymers and copolymers are preferred. For example, vinyl caprolactam and vinyl pyrrolidone are suitable vinyl lactams. Polyvinyl pyrrolidone, polyvinyl caprolactam and vinyl pyrrolidone/vinyl acetate copolymers are especially preferred. Luviskol®) VA 37 and Luviskol® VA 64 are especially preferred commercial products.

Suitable anionic hair-fixing polymers include synthetic homo- or copolymers with neutralizable monomer units containing acid groups, which are copolymerizable with comonomers, if necessary, which contain no acid groups. The acid groups may include —COOH, —SO₃H, —OSO₃H, —OPO₂H and —OPO₃H groups, of which the carboxylic acid groups are especially preferred. The acid groups can be unneutralized, or partially or completely neutralized. They are present in the composition according to the invention in a form that is 50% to 100% neutralized. As the neutralizing agent the above-mentioned neutralization agents can be used. Suitable monomers include unsaturated, radically polymerizable compounds, which have at least one acid group, especially carboxyvinyl monomers. Suitable monomers including acid groups are, e.g., acrylic acid, methacrylic acid, crotonic acid, maleic acid,r maleic acid anhydride or their monoesters, aldehydocarboxylic acid or ketocarboxylic acid.

Comonomers not substituted with acid groups include, e.g., acryl amide, methacrylamides, alkyl and dialkylacrylamides, alkyl and dialkylmethacrylamides, alkylacrylates, alkylmethacrylates, vinyl caprolactone, vinyl pyrrolidone, vinyl esters, vinyl alcohol, propylene glycol or ethylene glycol, amine-substituted vinyl monomers, such as dialkylaminoalkylacrylates, dialkylaminoalkylmethacrylates, monoalkylaminoalkylacrylates and monoalkylaminoalkylmethacrylates, in which the alkyl groups of these monomers preferably contain one to seven carbon atoms, especially preferably from one to three carbon atoms.

Suitable polymers with acid groups (different from those of ingredient A) include especially copolymers of acrylic acid or methacrylic acid with monomers selected from the group consisting of acrylic acid or methacrylic acid esters, acryl amides, methacrylamides and vinylpyrrolidones, homopolymers of crotonic acid and copolymers of crotonic acid with monomers selected from the group consisting of vinyl esters, acrylic acid or methacrylic acid esters, acrylamides and methacrylamides. A suitable natural polymer is, for example, shellac.

Preferred polymers with acid groups include cross-linked or uncross-linked vinyl acetate/crotonic acid copolymers. Similarly partially esterified copolymers between vinyl methyl ether and maleic acid anhydride are also preferred. Additional suitable anionic polymers include, e.g., terpolymers of acrylic acid, alkyl acrylate and N-alkylacrylamide, especially acrylic acid/ethyl acrylate/N-t-butylacrylamide terpolymer, terpolymers of vinyl acetate, crotonate and vinyl alkanoate, especially vinyl acetate/crotonate/vinyl neodecanoate copolymers.

Suitable film-forming amphoteric polymers are those polymers, which contain basic or cationic groups as additional functional groups besides acidic or anionic groups. The basic or cationic groups are, for example, primary, secondary, tertiary or quaternary amine groups. Examples include copolymers made from alkylacrylamides (especially octylacrylamide), alkylaminoalkylmethacrylates (especially t-butylaminoethylmethacrylate), and two or more monomers, comprising acrylic acid, methacrylic acid or their esters, wherein the alkyl groups contain from one to four carbon atoms, at least one of the monomers contains an acid group and which are obtained under the trademark Amphomer® or Amphomer® LV-71 of NATIONAL STARCH, USA.

Further examples of suitable hair-fixing polymers include copolymers of acrylic acid, methacrylate and methacrylamideopropyltrimethylammonium chloride (INCI: polyquaternium-47), copolymers made from acrylamidopropyltrimethyl ammonium chloride and acrylates, copolymers made from acrylamide, acrylamidopropyltrimethylammonium chloride, 2-amidopropylacrylamide sulfonate and dimethylaminopropylamine (INCI: Polyquaternium-43), or chitosans. Suitable polymers also comprise monomers carrying betaine groups, such as copolymers of methacryloylethylbetaine and two or more monomers made from acrylic acid or their simple esters, known under the INCI designation methacryloyl ethyl betaine/acrylates copolymer.

The composition according to the invention is preferably packaged in an aqueous, an alcoholic or aqueous/alcoholic medium, preferably with 10 percent by weight, especially preferably at least 50 percent water, and preferably a maximum amount of 40 percent by weight alcohol. Lower univalent alcohol suitable for cosmetic purposes and having from one to four carbon atoms, such as, e.g., ethanol or isopropanol, can be contained as the alcohol in the compositions according to the invention. The compositions according to the invention preferably has a pH of at least 6, especially preferably of at least 6.5. With low pH values the neutralization of the acid groups of the gel former is not sufficient and the consistency is too thin. A pH range between 6.0 and 8.0 is especially preferred. In a preferred embodiment the gel contains multivalent alcohol, preferably those with 2 to 5 carbon atoms and with 2 to 5 hydroxy groups in an amount of from 0.1 to 15, preferably from 1 to 10, percent by weight. Glycerol, ethylene glycol and propylene glycol, especially 1,2-propylene glycol, are especially preferred, as the multivalent alcohol.

The compositions according to the invention can also contain conventional additive ingredients suitable for hair treatment compositions. These additive ingredients include, e.g. wetting agents or emulsifiers from the classes of non-ionic, anionic, cationic or amphoteric surface-active agents, such as ethoxylated fatty alcohols, fatty alcohol sulfates, alkylbenzene sulfonates, alkyltrimethyl ammonium salts, alkyl betaines, in an amount of from 0.1 to 15 percent by weight; moisturizing agents; perfume oils, in an amount of 0.1 to 1 percent by weight; turbidity-inducing agents, such as ethylene glycol distearate, in an amount of about 0.2 to 5.0 percent by weight; pearlescence-imparting agents, such as a mixture of fatty acid monoalkylolamide and ethylene glycol distearate, in an amount of about 1.0 to 10 percent by weight; bactericidal and fungicidal agents, for example, 2,4,4-trichloro-2-hydroxydiphenyl ether or methyl chloroisothiazolione, in an amount of from 0.01 to 1.0 percent by weight; buffer substances, such as sodium citrate or sodium phosphate, in an amount of from 0.1 to 1.0 percent by weight; dyestuffs, for example, fluorescein sodium salt, in an amount of about 0.1 to 1.0 percent by weight; care materials, such as betaine, panthenol, plant and vegetable extracts, protein and silk hydrolyzates, lanolin derivative compounds, in an amount of 0.1 to 5 percent by weight, physiologically compatible silicone derivative compounds, such as volatile or non-volatile silicone oils or high-molecular-weight siloxane polymers in an amount of from 0.05 to 20 percent by weight; light protective materials, antioxidants, radical trapping agents, anti-flaking active ingredients, in an amount of about 0.01 to 4 percent by weight; fatty alcohols, luster-imparting ingredients, vitamins, softening agents, combability improving agents, de-fatting agents and anti-foaming ingredients.

The composition according to the invention is preferably in the form of a clear, transparent or at least translucent gel. The gel can be colored or colorless.

The composition according to the invention is characterized by especially positive spraying properties, especially when it is sprayed by means of a mechanically operated spraying apparatus. The subject matter according to the invention is thus a hair spray product, which comprises a container provided with spray pump and a thickened, gel-form composition containing at least one gel former, which is selected from

homopolymers and copolymers built up from at least one monomer, which is an acryl- or methacrylamidoalkylsulfonic acid or a salt thereof, and

thickening acrylate polymers made from acrylic or methacrylic acid and at least one acrylic or methacrylic acid ester, wherein the acrylate polymers are selected from those acrylate polymers, which provide a viscosity, in 1% aqueous solution, after adjusting pH to 7.5 with sodium hydroxide, in a range of 2000 to 8000 mPa·s (20° C., Brookfield RVT, 20 U/min).

Suitable gel formers, fixing polymers and compositions are described in detail above. Preferred spray pumps are those, which have an operating force of at maximum 40 N, preferably 10 to 30 N, especially preferably of 12 to 25 N at a delivery rate of from 0.1 to 0.3 ml, preferably from 0.15 to 0.25 ml per stroke.

The composition according to the invention forms a satisfactory spray and is sufficiently distributable on the hair. It does not load the hair and thus is also suitable for fine hair. It provides a good hold for a hairstyle as a hair-fixing agent, making the hair sticky or loading the hair. Fine hair becomes full and voluminous. As a hair-fixing gel the hair treatment composition provides improved fixing properties and especially a clearly improved luster in comparison to commercial gels formulated on a carbomer basis.

The following examples should clearly illustrate the subject matter according to the invention.

### EXAMPLES

### Example 1

### Hair Gels

| GEL | A | | B | | C | |
|---|---|---|---|---|---|---|
| Carbopol ® Aqua SF1 | 3.0 | g | — | | — | |
| (Acrylates Copolymer, 30% in | | | | | | |
| water) | | | | | | |
| Ammonium Acryloyldi- | — | | 0.4 | g | — | |
| methyl-Taurate/VP copolymer | | | | | | |
| Carbopol ® 980 (Polyacrylic | — | | — | | 0.175 | g |
| acid) | | | | | | |
| PVP/VA Copolymer | 2.5 | g | 2.5 | g | 2.5 | g |
| Aminomethylpropanol | 1.425 | g | 0.0038 | g | 0.095 | g |
| PPG-1-PEG-9 Lauryl Glycol | 0.2 | g | 0.2 | g | 0.2 | g |
| Ether | | | | | | |
| PEG-40 Hydrogenated Castor | 0.18 | g | 0.18 | g | 0.18 | g |
| Oil | | | | | | |
| Perfume | 0.2 | g | 0.2 | g | 0.2 | g |
| Ethanol | 31.0 | g | 31.0 | g | 31.0 | g |
| Water | To 100 | g | To 100 | g | To 100 | g |

Three hair gels of comparable viscosity were made using different gel formers. The amount of gel former was selected so that the gels have about equal viscosity and equal consistency. The gel compositions were compared in regarding the their effectiveness in providing luster to treated hair, hair fixing and droplet size distribution provided during spraying. The gels A and B are of the invention. Gel C is a commercial gel composition formulated on a carbomer basis.

Luster

Hair locks or skeins were wound around gray PVC rollers (width 50 mm, diameter 75 mm) and fixed on them with rubber bands to prepare for measuring luster. The rollers were fixed on a stand so that the locks were vertically oriented from roots to tips. The respective locks were uniformly sprayed with corresponding compositions by means of four pump strokes from a distance of 10 to 12 cm and subsequently dried to room temperature. For the luster measurement a parallel light beam was directed at the hair lock surface and the angular distribution of the reflected light and the light scattered in and under the surface was measured. The higher the proportion of directly reflected light and the less the portion of scattered light, the higher the luster. A luster classification can occur by measurement and evaluation of the angular distribution of the light returning from the surface. A narrower angular distribution (smaller half width HWB) means that luster has increased. A wider angular distribution (greater half width HWB) means that luster has decreased. The measurement of the angular distribution occurs with a digital camera, the data are read into a computer and evaluated with image processing software (Optimate 5.2). The procedure included two luster measurements per hair lock or hair sample and three hair samples or hair locks for each composition, which means six measurements per each composition. The average value of the individual values was calculated.

Results of the Luster Measurements:

| | |
|---|---|
| Untreated Locks | HWB = 42° |
| Gel A of the invention: | HWB = 35° |
| Gel B of the invention: | HWB = 36° |
| Gel C of the prior art: | HWB = 39° |

The replacement of the thickener of hair gel C by either thickner of Gel A or Gel B leads surprisingly to an increase in the hair luster. The measured results correlate with judgements of hair quality, including luster, made by a panel of hair styling experts based on visual observations.

Hair Fixing

The comparison of the fixing power occurred by measuring the breaking force. For this purpose standard, unbleached hair locks (100 hairs/lock) were washed with a standard shampoo, rinsed with water and dried. Three locks for each different composition were laid in a foil pocket and wet with water. Subsequently a total of 90 microliters of each of the compositions to be tested (30 microliters per lock) were applied. The foil packets were closed and after an acting time of 10 minutes all locks were dried overnight on a smooth non-absorbing bed in a climate-controlled room at 20° C. and 65% relative humidity. After that nine breaking force values were measured for each stand (3 measurements at the start, in the center and ends of the locks). The breaking force is a measure for the hair fixing.

The measurements were performed with a breaking force measurement apparatus, with which the dried locks are subjected to a three-point bending test. The hair lock or skein is supported at two points (on a support) and loaded in the center with a force sensor. A computer records the bending force as a function of the extent of bending in the form of a diagram. The greater the maximum bending force (=breaking force), the greater is the fixing action of the product. The maximum bending force is designated as the breaking force, since at this point the polymeric compounds (e.g. the polymer film) between the hairs that provides the fixing action breaks. Three measurements per stand and three lock samples per composition, i.e. a total of nine measurements for each tested gel composition A, B or C, were performed and the average values were calculated.

Results of the Breaking Force Measurements:

| | |
|---|---|
| Gel composition A of the invention: | 0.30 N |
| Gel composition B of the invention: | 0.26 N |
| Gel composition C of the prior art: | 0.19 N. |

The replacement of the thickener of the prior art with that of Gel A or Gel B surprisingly leads to an increase in the hair fixing power.

Spray Droplet Size Distribution

To measure the inhalable portion of the spray with a diameter less than 10 μm during spraying of the compositions, the compositions were sprayed with a spray pump (Coster GMSP 38/200, Nozzle V 06.233). The drop size distributions for the compositions were measured with the help of a particle measuring apparatus operating on the basis of light scattering (Malvern Particle Sizer).

Portion of Spray Droplets with a Diameter of Less Than 10 μm

| | |
|---|---|
| Gel composition A of the invention: | 0.65% |
| Gel composition B of the invention: | 0.7% |
| Gel composition C of the prior art: | 1.0% |

The disclosure in German Patent Application P101 63 500.1 of Dec. 21, 2001 is incorporated here by reference. This German Patent Application describes the invention described hereinabove and claimed in the claims appended hereinbelow and provides the basis for a claim of priority for the instant invention under 35 U.S.C. 119.

While the invention has been illustrated and described as embodied in sprayable hair gels, it is not intended to be limited to the details shown, since various modifications and changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed is new and is set forth in the following appended claims.

We claim:

1. A hair gel composition containing a combination of at least one hair fixing polymer and at least one gel former, said at least one gel former being selected from the group consisting of homopolymers, copolymers and thickening acrylate polymers; wherein the homopolymers and the copolymers are built up from at least one monomer, and said at least one monomer is selected from the group consisting of acrylamidoalkylsulfonic acids, methacrylamidoalkylsulfonic acids and salts thereof, and the thickening acrylate polymers are made from acrylic acid or methacrylic acid and at least one acrylic acid ester or methacrylic acid ester, wherein 1% aqueous solutions of said thickening acrylate polymers, after adjusting pH thereof to 7.5 with sodium hydroxide, have viscosities in a range of 2000 to 8000 mPa·s, at 20° C., Brookfield RVT, and at 20 rev/min.

2. The hair gel composition as defined in claim 1, having a viscosity from 100 to 5000 mPa·s, measured as a shear viscosity with a Bohlin Rheometer CS, measuring body C25, at a temperature of 25° C. and a shear rate of 50 s⁻¹.

3. The hair gel composition as defined in claim 1, containing said at least one gel former in an amount of from 0.1 to 10 percent by weight and said at least one hair-fixing polymer in an amount of from 0.1 to 15 percent by weight.

4. The hair gel composition as defined in claim 1, wherein said at least one monomer is of formula H₂C═CH−C(═O)—NH-A-SO₃H or is a salt thereof, wherein A represents a divalent C₂- to C₆-hydrocarbon group.

5. The hair gel composition as defined in claim 1, wherein the at least one gel former is an ammonium acryloyldimethyltaurate/vinyl pyrrolidone copolymer.

6. The hair gel composition as defined in claim 1, wherein the at least one hair-fixing polymer is a non-ionic vinyl lactam homopolymer or copolymer.

7. A hair spray product, said hair spray product comprising a container provided with a spray pump and a thickened gel-form composition, said thickened gel-form composition containing at least one hair-fixing polymer and at least one gel-former; wherein said at least one gel former is selected from the group consisting of homopolymers, copolymers and thickening acrylate polymers; wherein the homopolymers and the copolymers are built up from at least one monomer, and said at least one monomer is selected from the group consisting of acrylamidoalkylsulfonic acids, methacryl-amidoalkylsulfonic acids and salts thereof, and the thickening acrylate polymers are made from acrylic acid or methacrylic acid and at least one acrylic acid ester or methacrylic acid ester, wherein 1% aqueous solutions of said thickening acrylate polymers, after adjusting pH thereof to 7.5 with sodium hydroxide, have viscosities in a range of 2000 to 8000 mPa·s, at 20° C., Brookfield RVT, and at 20 rev/min.

8. The hair spray product as defined in claim 7, wherein said gel-form composition contains said at least one gel former in an amount of from 0.1 to 10 percent by weight and said at least one hair-fixing polymer in an amount of from 0.1 to 15 percent by weight and said gel-form composition has a viscosity from 100 to 5000 mPa·s, measured as a shear viscosity with a Bohlin Rheometer CS, measuring body C25, at a temperature of 25° C. and a shear rate of 50 s⁻¹.

9. The hair spray product as defined in claim 7, wherein said at least one monomer is of formula H₂C═CH—C(═O)—NH-A-SO₃H or is a salt thereof, wherein A represents a divalent C₂- to C₆-hydrocarbon group.

10. The hair spray product as defined in claim 7, wherein the at least one gel former is an ammonium acryloyldimethyltaurate/vinyl pyrrolidone copolymer.

11. The hair spray product as defined in claim 7, wherein the at least one hair-fixing polymer is a non-ionic vinyl lactam homopolymer or copolymer.

12. The hair spray product as defined in claim 7, wherein said spray pump is a mechanically operable pump that produces an operating force of from 10 to 40 N at a delivery rate of 0.1 to 0.3 ml per stroke.

13. A method of increasing luster or hold of a hair style, said method comprising the step of applying at least one polymer to the hair, wherein said at least one polymer is
a homopolymer or copolymer built up from at least one monomer, and said at least one monomer is selected from the group consisting of acrylamidoalkylsulfonic acids, methacrylamidoalkylsulfonic acids and salts thereof, or
a thickening acrylate polymer made from acrylic acid or methacrylic acid and at least one acrylic or methacrylic acid ester, whose 1% aqueous solutions, after adjusting pH thereof to 7.5 with sodium hydroxide, have viscosities in a range of 2000 to 8000 mPa·s, at 20° C., Brookfield RVT, and at 20 rev/min.

14. The method as defined in claim 13, wherein said at least one monomer is of formula H₂C═CH—C(═O)—NH-A-SO₃H or is a salt thereof, wherein A represents a divalent C₂- to C₆-hydrocarbon group.
